(19) United States
(12) Patent Application Publication
(10) Pub. No.: US 2023/0218125 A1
(43) Pub. Date: Jul. 13, 2023

(54) MULTIFUNCTIONAL DRYER STRUCTURE

(71) Applicant: Guangdong Willing Technology Corporation, Huizhou (CN)

(72) Inventor: JINGNONG YE, Huizhou (CN)

(21) Appl. No.: 17/574,558

(22) Filed: Jan. 13, 2022

(51) Int. Cl. A47K 10/06; A47K 10/48; A61L 2/20; A61L 2/10

(52) U.S. Cl. CPC A47K 10/06; A47K 10/48; A61L 2/202; A61L 2/10; A61L 2202/26

(57) ABSTRACT

A multifunctional dryer structure includes a first drying mechanism, at least one connecting element configured on one side of the first drying mechanism, a second drying mechanism configured on another side of the connecting element, at least one hanging element configured on one side of the first drying mechanism, and a control module. The first drying mechanism is provided with a first air outlet and UV ozone lamp at one side of the hanging element, and the second drying mechanism is formed with a second air outlet and has a sensing element and control module in electric connection with the UV ozone lamp. With the above structure, the second drying mechanism is used for foot drying, first drying mechanism towel drying, and UV ozone lamp towel disinfection, thereby achieving the purpose of multi-machine integration, and drying wet feet and wet towels quickly after taking a bath.

### (A) TECHNICAL FIELD OF THE INVENTION

The present invention relates to a smart small appliance, and more particularly to a multifunctional dryer structure having the functions of foot drying, towel drying and towel disinfection, and can automatically and safely perform drying and disinfection actions.

### (B) DESCRIPTION OF THE PRIOR ART

After bathing, most people usually use a dry towel to dry the body water. However, the towel has already absorbed part of body's moisture and is soaked after the body is wiped. Although the wet towel can be wrung dry and then continue to be used to wipe the body, the action of wringing the towel cannot completely dry, so the towel is still damp, and it is impossible to completely dry the body, such that the towel is still wet, unable to wipe the body completely dry. Therefore, when leaving the bathroom, not only the towels are still damp, but the body is not completely dry, especially the parts of the foot soles and finger seams.

People always choose to dry naturally or use a towel to dry the moisture on the feet; upon natural dry, the moisture on the feet needs a longer time to be dried, and it is difficult to dry naturally between the toes; if a towel is used to dry the moisture on the feet, first of all, because the bathroom is usually a relatively humid environment, the wet towel is hung in the bathroom to dry, and the towel itself being dry enough or not is a problem, and secondly, if the towel is not dried or disinfected in time, then the towel will be contaminated with bacteria or odor, and when it is wet or placed for a long time, it is easy to breed bacteria and produce odor, which will cause foot infections when the towel is used next time.

### SUMMARY OF THE INVENTION

To design a multifunctional dryer structure having foot drying, towel drying and towel disinfection effects, and capable of automatically and safely performing drying and disinfection, the present invention is proposed.

The main object of the present invention is to provide a multifunctional dryer structure, combining a first drying mechanism for towel drying, a second drying mechanism for foot drying and a ultraviolet ozone lamp into one body, so as to be acted as a smart small appliance, thereby having use convenience.

To achieve the above object, the present invention proposes a multifunctional dryer structure, including: a first dryer mechanism; at least one connecting element, configured on one side of the first drying mechanism; a second drying mechanism, configured on one side of the connecting element away from the first drying mechanism; at least one hanging element, configured on one side of the first drying mechanism; a first air outlet, formed on the first drying mechanism and positioned at one side of the hanging element; a ultraviolet ozone lamp, configured on the first drying mechanism and positioned at one side of the hanging element; a sensing element, in electric connection with the ultraviolet ozone lame adapted to close the ultraviolet ozone lamp when a human body is sensed; a second air outlet, formed on the second drying mechanism for drying the feet of the human body; and a control module, in electric connection with the first drying mechanism, second drying mechanism, ultraviolet ozone lamp and sensing element.

In use, towels can be hung on the hanging element to dry, and a user only needs to operate the control module to start the first drying mechanism and second drying mechanism simultaneously, so as to dry the towels and the feet. In addition, when the sensing element judges that people is leaving, the ultraviolet ozone lamp is started to disinfect the towels. In this way, the three-in-one design not only simplifies the structure and improves the convenience, but also takes into account the safety of use.

With the above technology, the present invention can breakthrough the disadvantages of being not easy to air dry, and easy to breed bacteria due to wet towels and feet, achieving the practical progressiveness of the above advantages.

### BRIEF DESCRIPTION OF THE DRAWINGS

FIG. 1 is a perspective view of a preferred embodiment of the present invention;

FIG. 2 is a perspective view of a first drying mechanism of the embodiment of the present invention viewing from another angle;

FIG. 3 is a perspective view of the embodiment of the present invention upon drying;

FIG. 4 is a perspective view of the embodiment of the present invention upon disinfection;

FIG. 5 is a perspective view of another preferred embodiment of the present invention;

FIG. 6 is a perspective view of still another preferred embodiment of the present invention upon adjustment;

FIG. 7 is a perspective view of the embodiment of the present invention, where dumping is prevented;

FIG. 8 is a perspective view of a further preferred embodiment of the present invention; and

FIG. 9 is a perspective view of the further embodiment of the present invention in a storage state.

### DETAILED DESCRIPTION OF THE PREFERRED EMBODIMENTS

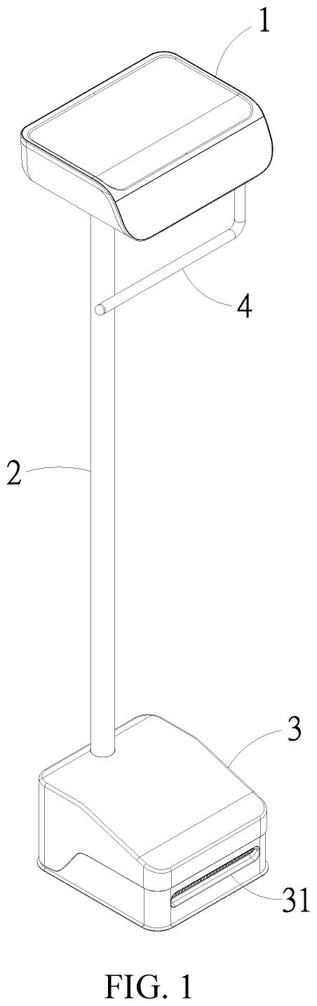

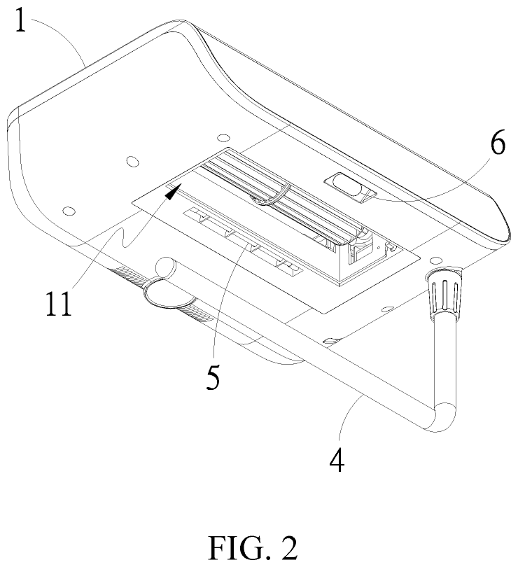

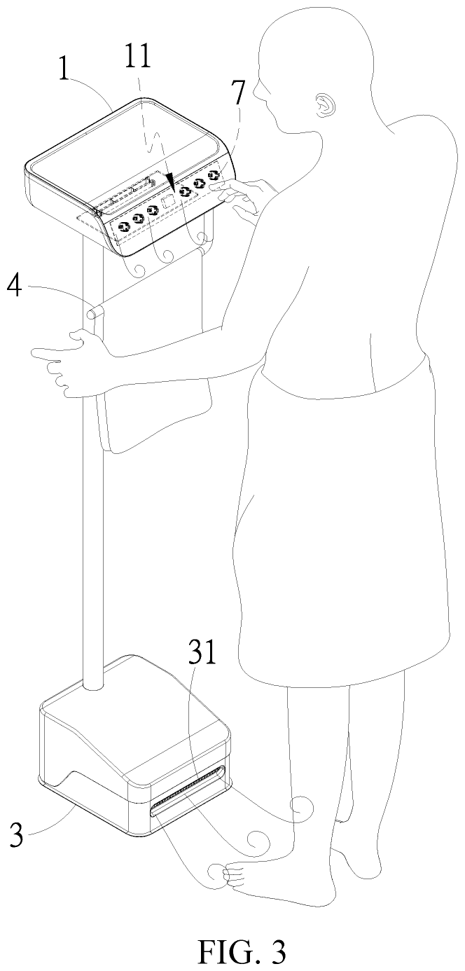

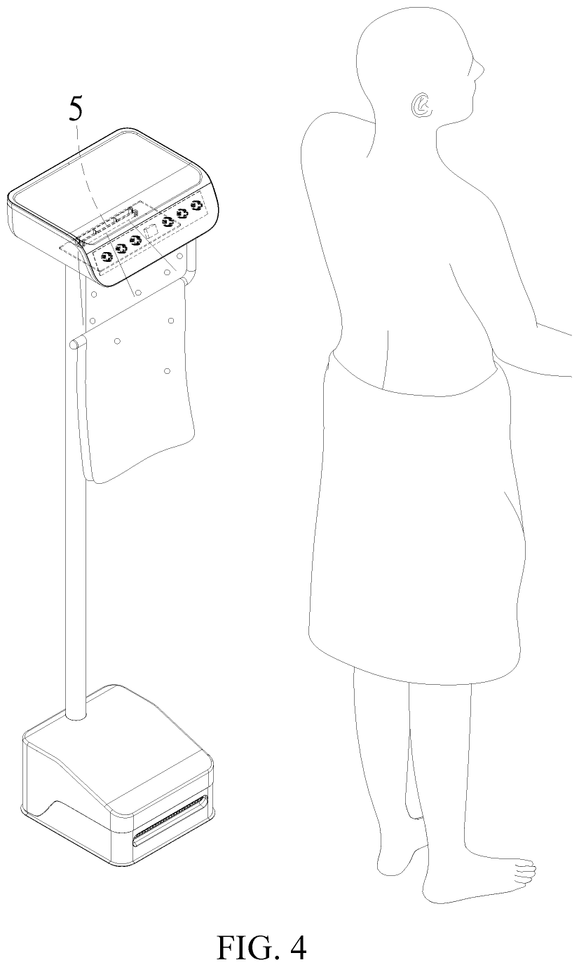

Referring to FIGS. 1 to 4, a multifunctional dryer structure of the present invention includes a first drying mechanism 1, at least one connecting element 2, a second drying mechanism 3, at least one hanging element 4, a first air outlet 4, a ultraviolet ozone lamp 5, a sensing element 6, a second air outlet 31, and a control module 7.

The at least one connecting element 2 is configured on one side of the first drying mechanism 1.

The second drying mechanism 3 is configured on one side of the connecting element away from the first drying mechanism 1.

The at least one hanging element 4 is configured on one side of the first drying mechanism 1.

The first air outlet 11 is formed on the first drying mechanism 1 and positioned at one side of the hanging element 4.

The ultraviolet ozone lamp 5 is configured on the first drying mechanism 1 and positioned at one side of the hanging element 4.

The sensing element 6 is in electric connection with the ultraviolet ozone lamp 5, and adapted to close the ultraviolet ozone lamp 5 when sensing a human body approaching, the present embodiment takes an infrared sensor as an example.

The second air outlet 31 is formed on the second drying mechanism 3, thereby drying the feet of the human body.

The control module 7 is in electric connection with the first drying mechanism 1, second drying mechanism 3, ultraviolet ozone lamp 5 and sensing element 6.

With the above components, it can be clearly seen that the present invention mainly combines the first drying mechanism 1 for drying towels and the second drying mechanism 3 for drying feet into one body; the present invention uses a single rod typed connecting element 2 to be in connection with the first drying mechanism 1 upward and the second drying mechanism 3 downward. In addition, the control module 7 is a control component at least having a circuit board, in this embodiment, a touch panel provided on the first drying mechanism 1 is taken as an example, and the sensing element 6 is also provided on the first drying mechanism 1.

In practical use, the hanging element 4 allows for the hanging of towels to be dried, a user only needs to operate the control module 7, and the first drying mechanism 1 and second drying mechanism 3 can then be started so as to blow out heat air from the first air outlet 11 on the lower side of the first drying mechanism 1 and the second air outlet 13 on the front side of the second drying mechanism 3 to dry the towels and feet simultaneously. The control module 7 has basic functions such as timing and temperature control, and the ultraviolet ozone lamp 5 is allowed to be started by the control module 7 by the control module 7 to disinfect the towels when no human body is sensed by the sensing elements 6, so as to ensure use safety. Furthermore, the ultraviolet ozone lamp 5 of the present invention uses light with wavelength 185 mm, and nitrogen dioxide in the air reacts into nitrogen monoxide and oxygen atoms after being irradiated, and oxygen atoms easily react with oxygen to form ozone (as shown by the small circle in the figures), so ozone can be used to diffuse to places where light cannot be seen to perform sterilization.

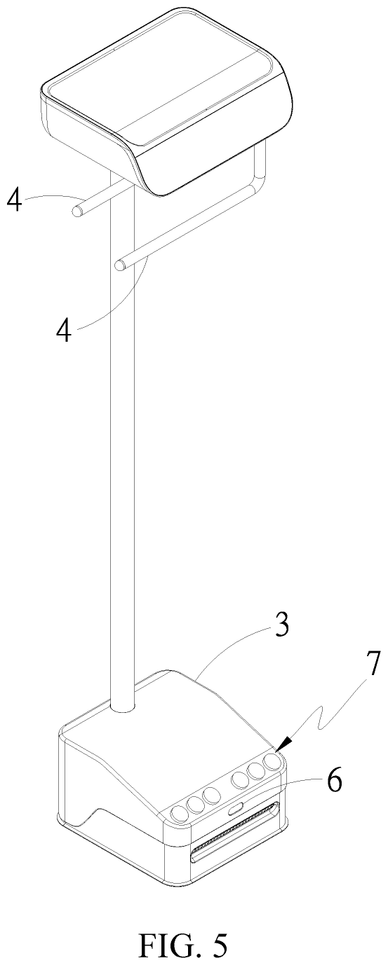

Referring to FIG. 5, showing another preferred embodiment of the present invention, it can be clearly seen that the present embodiment is almost the same as the above embodiment except that in the present invention, the control module 7 and sensing elements 6 are coupled to the second drying mechanism 3, and the control module 7 takes rotary switches as an example. In addition, the number of the hanging elements 4 is changed to two, so also having the functions such as towel drying, feet drying and towel sterilization.

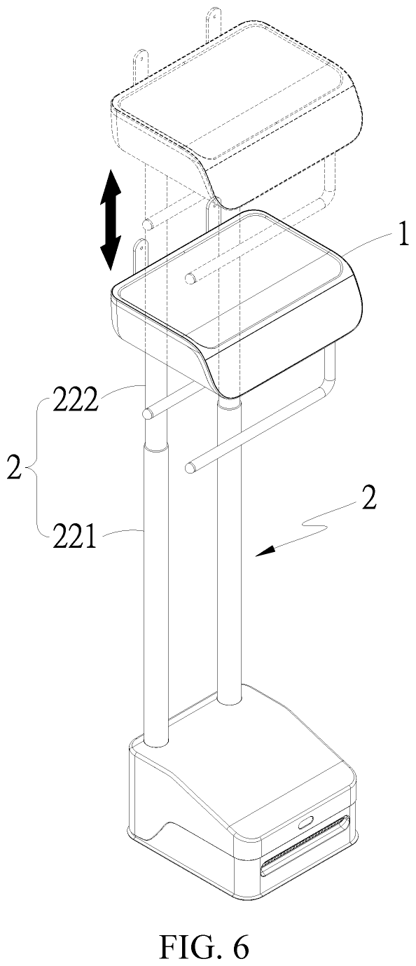

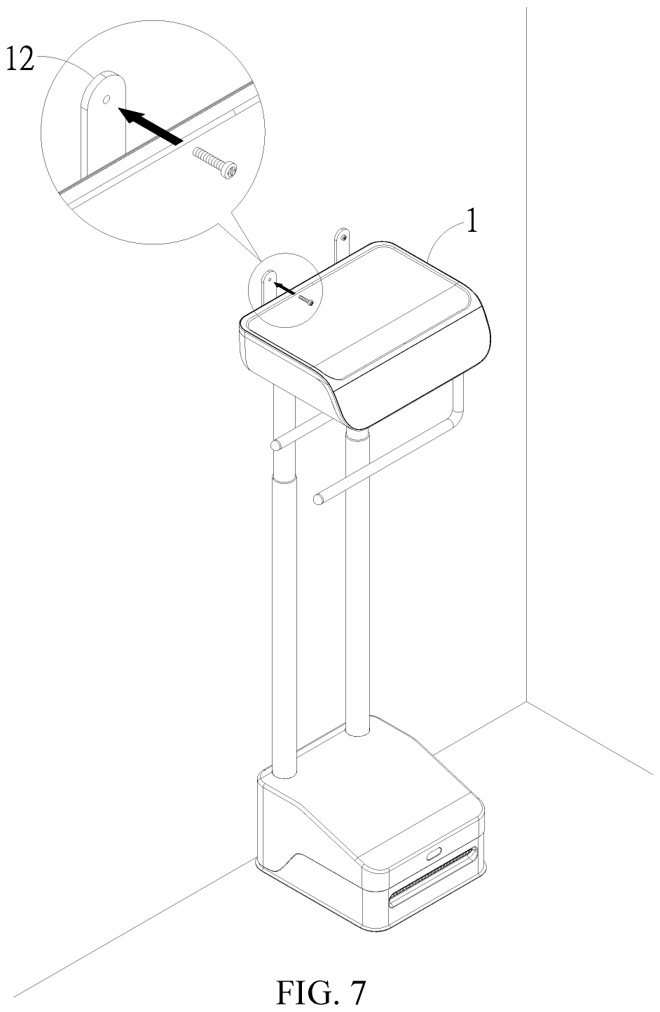

Referring to FIGS. 6 and 7, showing still another preferred embodiment, it can be clearly seen that the embodiment is almost similar to the above embodiments except that in the embodiment, one side of the first drying mechanism 1 is provided with at least on positioning element 12, where the positioning element 12 is exemplified as a locking sheet, and the positioning element 12 is used to fix the dryer of the present invention to a wall surface, preventing it from dumping. If there is a nail on the wall surface, the positioning element 12 can be used as a hanging hook. Furthermore, the number of the connection elements 2 is increased to two, where each connecting element 2 has a first telescopic rod 221 and a second telescopic rod 222 movably configured on the first telescopic rod 221, allowing users to adjust the height of the first drying mechanism 1 freely.

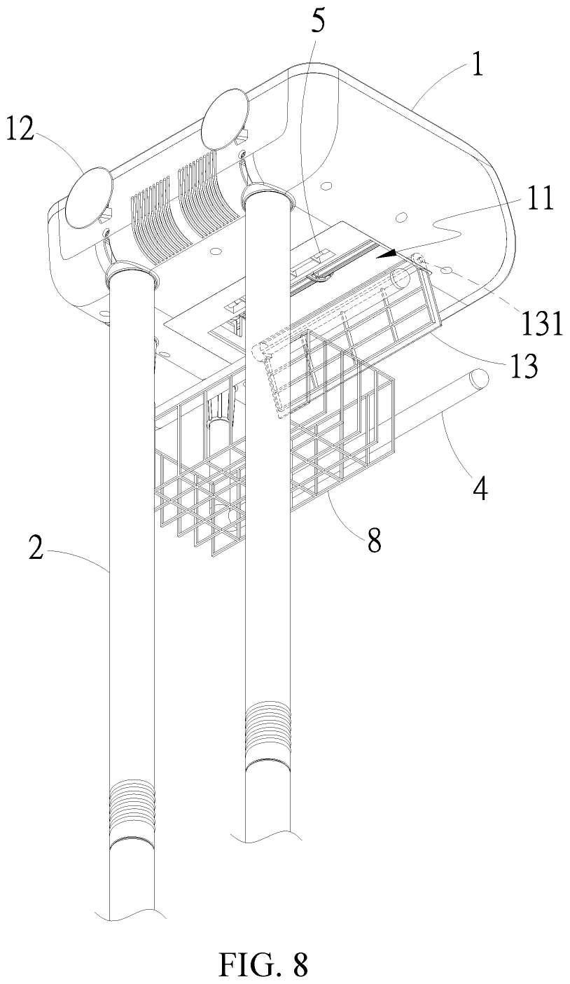

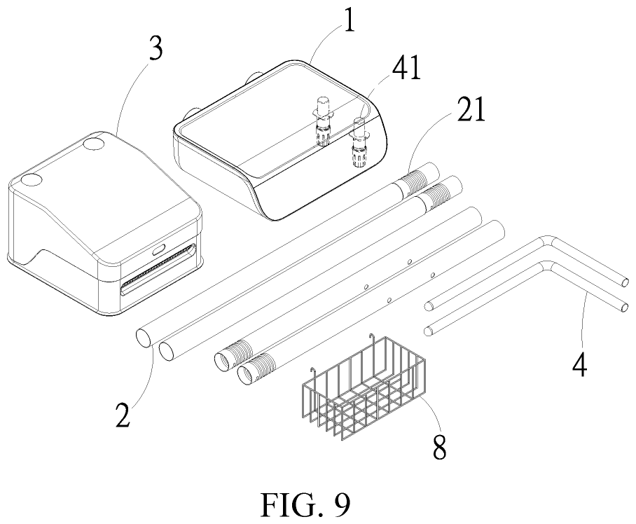

Referring to FIGS. 8 and 9, showing a further preferred embodiment, it can be clearly seen from the figures that the present embodiment is almost similar to the above embodiments except that in the present invention, the positioning element 12 is changed to a sucker type. Since general bathroom wall surfaces are smooth surfaces, the sucker is more convenient for fixing, and can be used to fix dryer without damaging the wall surface. Furthermore, the first air outlet 11 is provided with a cover plate 13 coupled pivotally to the first air outlet 11 through a rotating shaft 13, where the cover plat 13 can be used to close the first drying mechanism 1 and ultraviolet ozone lamp 5 when they are not used, thereby having dustproof and moisture-proof effects. Furthermore, one side of the hanging element 4 is provided with at least one placement element 8 hung on the connecting element 2 capable of allowing users to place clothes thereon. Furthermore, the connecting element 2 and hanging element 4 are respectively provided with an assembly element 21, 41 so as to allow detachable assembly. Whereby, the first drying mechanism 1, second drying mechanism 3, connecting element 2, hanging element 4 and placement element 8 can be detached and separated, so as to facilitate packaging and shipping, and at the same time, have structurally simple and cost decreasing effects.

I claim:

1. A multifunctional dryer structure, comprising:
a first dryer mechanism;
at least one connecting element, configured on one side of said first drying mechanism;
a second drying mechanism, configured on one side of said connecting element away from said first drying mechanism;
at least one hanging element, configured on one side of said first drying mechanism;
a first air outlet, formed on said first drying mechanism and positioned at one side of said hanging element;
a ultraviolet ozone lamp, configured on said first drying mechanism and positioned at one side of said hanging element;
a sensing element, in electric connection with said ultraviolet ozone lame adapted to close said ultraviolet ozone lamp when a human body is sensed;
a second air outlet, formed on said second drying mechanism for drying the feet of said human body; and
a control module, in electric connection with said first drying mechanism, second drying mechanism, ultraviolet ozone lamp and sensing element.

2. The structure according to claim 2, wherein one side of said hanging element is provided with at least one placement element.

3. The structure according to claim 1, wherein said first air outlet is provided with a cover plate.

4. The structure according to claim 3, wherein said cover plate is coupled to said first air outlet through a rotating shaft.

5. The structure according to claim 1, wherein said at least one positioning element I configured on one side of said first drying mechanism away from said control module.

6. The structure according to claim 5, wherein said positioning element is sucker, hanging hook or locking sheet.

7. The structure according to claim 1, wherein said connecting element has a first telescopic rod, and a second telescopic rod movably configured on said first telescopic rod.

8. The structure according to claim 1, wherein said connecting element and hanging element respectively has an assembly element for performing detachable assembly.
